(19) United States
(12) Patent Application Publication
(10) Pub. No.: US 2024/0277598 A1
(43) Pub. Date: Aug. 22, 2024

(54) SILICONE-FREE CONDITIONING SHAMPOO COMPOSITION

(71) Applicant: The Procter & Gamble Company, Cincinnati, OH (US)

(72) Inventors: Mark Anthony Brown, Union, KY (US); John Samuel Kenney, JR., Cincinnati, OH (US)

(21) Appl. No.: 18/435,019

(22) Filed: Feb. 7, 2024

Related U.S. Application Data
(60) Provisional application No. 63/444,737, filed on Feb. 10, 2023.

(51) Int. Cl. A61K 8/81; A61K 8/04; A61K 8/34; A61K 8/44; A61K 8/46; A61Q 5/12

(52) U.S. Cl. CPC A61K 8/8129; A61K 8/042; A61K 8/342; A61K 8/44; A61K 8/466; A61Q 5/12; A61K 2800/34

(57) ABSTRACT

A liquid conditioning shampoo composition that is free or substantially free of silicone. The shampoo can contain a detersive surfactant, polyvinyl alcohol, a gel network, and a liquid carrier. The gel network contains a gel network surfactant, a fatty alcohol, and a liquid carrier. The conditioning shampoo composition can have similar performance characteristics to a conditioning shampoo that contains silicone.

### FIELD OF THE INVENTION

The present invention generally relates to conditioning shampoo compositions that are free of silicone, and more specifically to conditioning shampoo compositions that contain polyvinyl alcohol, one or more cationic polymers, and a gel network that provide similar conditioning performance as compared to shampoos that contain silicone.

### BACKGROUND OF THE INVENTION

Human hair becomes soiled due to its contact with the surrounding environment and from the sebum secreted by the scalp, in addition to the use of third-step stylers and hair treatments that leave a visually or tactilely noticeable residue behind on the hair. The soiling of hair causes it to have a dirty feel and an unattractive appearance, necessitating regular shampooing.

Shampooing cleans the hair by removing excess soil and sebum. However, shampooing can leave the hair in a wet, tangled, and generally unmanageable state. Once the hair dries, it is often left in a dry, rough, lusterless, and/or frizzy condition due to removal of the hair's natural oils. Therefore, shampoos that provide a cleansing and conditioning benefit to hair (hereinafter “conditioning shampoos”) are popular. Conditioning shampoos frequently contain silicones that coat the hair shaft, thereby locking in moisture, which reduces frizz and gives hair a coveted soft and silky feel. Silicones can provide these conditioning benefits while not interfering with cleansing efficacy.

However, some consumers prefer hair care products that are silicone-free. Many alternatives, such as natural oils, have been incorporated into shampoo compositions. However, these alternatives, are generally less effective and/or leave the hair looking and feeling greasy and/or dirty.

Therefore, there is a need for a silicone-free shampoo composition that provides good conditioning and cleaning benefits.

### SUMMARY OF THE INVENTION

A conditioning shampoo composition comprising: (a) about 4% to about 25% of a detersive surfactant; (b) about 0.05% to about 5% of a polyvinyl alcohol; (c) about 1% to about 8% of a fatty alcohol; (d) about 0.01% to about 15% of one or more gel network surfactants; wherein the one or more gel network surfactants comprise an anionic surfactants, cationic surfactants, zwitterionic surfactants, non-ionic surfactants, or a combination thereof; (e) about 60% to about 85% of a liquid carrier; (f) a dispersed gel network phase comprising: (i) at least a portion of the fatty alcohol; (ii) at least a portion of the gel network surfactant; (iii) at least a portion of the liquid carrier; wherein the anionic surfactant and the gel network surfactant are the same or different; wherein the composition is substantially free of silicone; wherein the composition is a liquid.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1A shows a hair tress washed with Comparative Example C5;

FIG. 1B shows a hair tress washed with Example 1;

FIG. 2A is an are image from a scanning electron microscope (SEM) of a strand of hair at 1000x that was washed with Comparative Example C5;

FIG. 2B is an image from a scanning electron microscope (SEM) of a strand of hair at 5000x that was washed with Comparative Example C5;

FIG. 2C is an image from a scanning electron microscope (SEM) of a strand of hair at 1000x that was washed with Example 1; and

FIG. 2D is an image from a scanning electron microscope (SEM) of a strand of hair at 5000x that was washed with Example 1.

### DETAILED DESCRIPTION OF THE INVENTION

Conditioning shampoo compositions comprising various combinations of detersive surfactant and hair conditioning agents are known. Many of these products comprise a combination of anionic surfactant, cationic polymer and silicone that work in combination to form coacervate, which deposits on hair during the wash, ultimately giving the hair a smooth texture and clean, silky feel.

However, some consumers want a liquid conditioning shampoo that is silicone-free. It is difficult to formulate a consumer acceptable conditioning shampoo that is silicone-free because silicone is a very effective hair conditioning agent, which makes it difficult to find a composition that matches its performance particularly the smooth, clean feeling of dry hair.

Emulsified, non-silicone conditioning actives, like coconut oil, are initially appealing silicone replacements. However, these actives partition into surfactant micelles within the aqueous phase over time, reducing product viscosity below what is typically consumer preferred, which makes it difficult to efficiently spread the product across a user's hair. Lamellar structured surfactant phases can be effective at stabilizing high levels (≥1%) of emulsified oils, however these products are typically slower to lather and generate less lather volume compared to conventional (micellar based) shampoos, which is generally not consumer preferred. Furthermore, when these actives are deposited at levels necessary to mimic silicone as a hair conditioning active, the dry hair generally looks and/or feels dirty, oily and/or greasy.

It was found that a liquid conditioning shampoo that contains polyvinyl alcohol (PVOH), one or more cationic polymers, and a gel network can meet or exceed the performance of silicone. Comparative Example C5 (see Table 2, hereafter) is a shampoo composition that contains 0.4% cationic polymer, 2.3% gel network premix, and 3% silicone. Example 1 (see Table 3, hereafter) is silicone-free and contains 0.4% cationic polymer, 2.3% gel network premix, and 0.5% polyvinyl alcohol. The silicone-free Example 1 had better dry hair performance, as determined by the Hair Texture Analysis Test Method, described hereafter, as compared to Comparative Example C5.

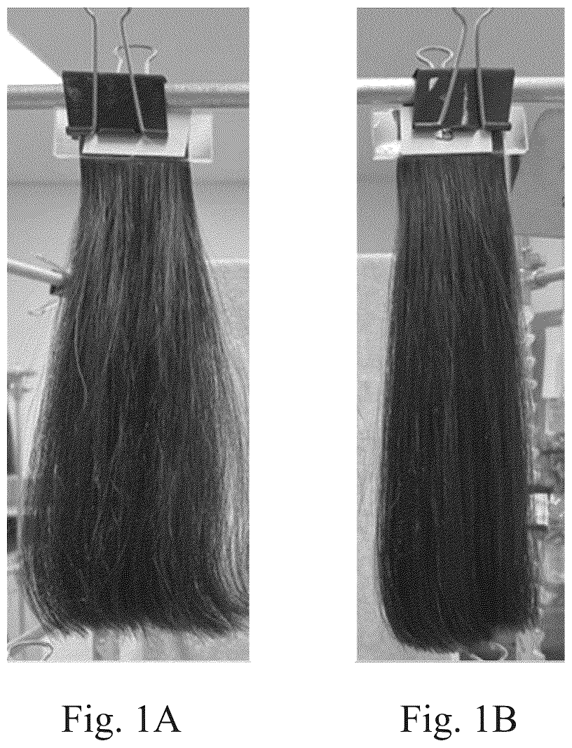

FIG. 1A shows a hair tress washed with Comparative Example C5 and FIG. 1B shows a hair tress washed with Example 1. Each tress was washed as described in the Hair Texture Analysis Test Method, described hereafter. Surprisingly, FIG. 1A, which was washed with a shampoo containing 3% silicone, clearly shows more frizz than Example 1, which was washed with a silicone-free shampoo that had 05% polyvinyl alcohol.

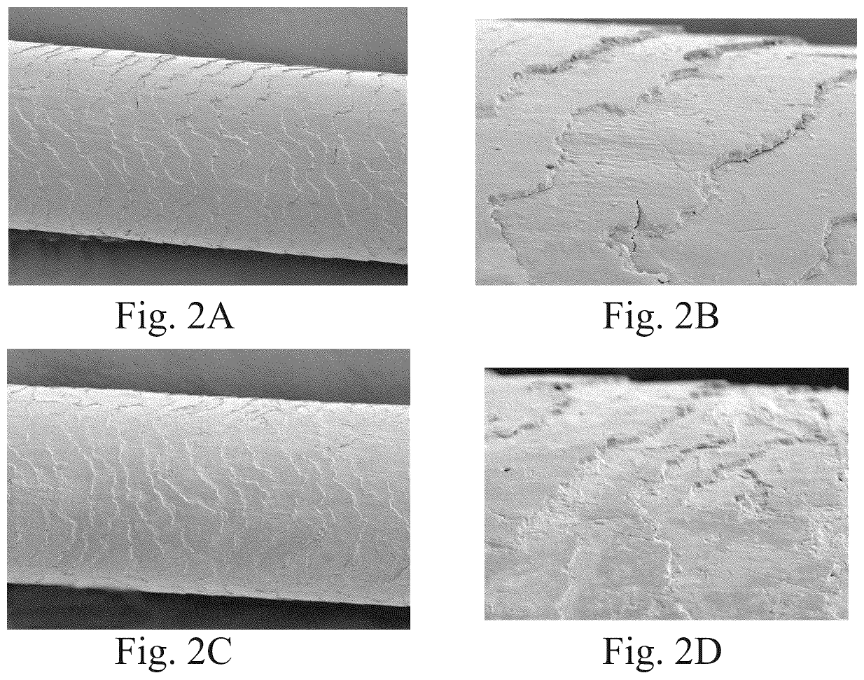

FIGS. 2A and 2B are images from a scanning electron microscope (SEM) of a strand of hair at 1000x and 5000x, respectively, that was washed with Comparative Example C5, the silicone containing shampoo, according to the Hair Texture Analysis Test Method, described hereafter. FIGS. 2C and 2D are SEM images of a strand of hair at 1000x and 5000x, respectively, that were washed with Example 1, the silicone-free shampoo that contained PVOH, according to the Hair Texture Analysis Test Method, described hereafter. Surprisingly, the strand in FIGS. 2A and 2B is rougher with well-defined ridges, as compared to the strand in FIGS. 2C and 2D.

The shampoo composition can have a viscosity of 2000 cP (2 Pa·s) to 23,000 cP (23 Pa·s), alternatively 4000 cP (4 Pa·s) to 14,000 cP (14 Pa·s), alternatively 4500 cP (4.5 Pa·s) to 12,000 cP (12 Pa·s), alternatively 5,000 cP (5 Pa·s) to 11,000 cP (11 Pa·s), and alternatively 7,000 cP (7 Pa·s) to 10,000 cP (10 Pa·s) as measured by the Cone/Plate Viscosity Measurement Test Method, described herein.

The shampoo composition can include 0.05% to 5% PVOH, alternatively 0.05% to 2% PVOH, alternatively 0.1% to 1.5% PVOH, alternatively 0.15% to 1% PVOH, alternatively 0.2% to 0.9% PVOH, and alternatively 0.2% to 0.5% PVOH. The PVOH can have a degree of hydrolysis >50%, alternatively >80%, alternatively >85%, and alternatively >90%. The PVOH can have a viscosity of 1 cP to 100 cP, alternatively 1 cP to 50 cP, alternatively 2 cP to 40 cP, alternatively 3 cP to 35 cP as measured as a 4% aqueous solution at 20° C. The PVOH Viscosity is measured according to the PVOH Viscosity test method, described herein.

A hair tress washed and dried according to the according to the Hair Texture Analysis Test Method, described hereafter, has an average detangling of less than 210 gf, alternatively less than 200 gf, alternatively less than 180 gf, alternatively less than 150 gf, alternatively less than 145 gf.

A hair tress washed and dried according to the Hair Texture Analysis Test Method, described hereafter, has an average detangling of 45 gf to 180 gf, alternatively 60 gf to 165 gf, alternatively 65 gf to 145 gf, alternatively 70 gf to 130 gf, and alternatively 70 gf to 100 gf.

A hair tress washed and dried according to the Hair Texture Analysis Test Method, described hereafter, has an average resistance at tips of less than 200 gf, alternatively less than 175 gf, alternatively less than 150 gf, alternatively less than 125 gf, and alternatively less than 110 gf. A hair tress washed and dried according to the Hair Texture Analysis Test Method, described hereafter, has an average resistance at tips of 25 gf to 120 gf, alternatively 35 gf to 110 gf, alternatively 40 gf to 105 gf, alternatively 40 gf to 95 gf, and alternatively 45 gf to 90 gf.

### Definitions

Reference within the specification to “embodiment(s)” or the like means that a particular material, feature, structure and/or characteristic described in connection with the embodiment is included in at least one embodiment, optionally several embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 21° C. and at ambient conditions, where “ambient conditions” means conditions under 1 atmosphere of pressure and at 50% relative humidity. All numeric ranges are inclusive of narrower ranges; delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. It is to be understood that, as used herein the terms “the,” “a,” or “an,” mean “at least one,” and should not be limited to “only one” unless explicitly indicated to the contrary.

“About” modifies a particular value by referring to a range of plus or minus 20% or less of the stated value (e.g., plus or minus 15% or less, 10% or less, 5% or less, or even 3% or less). “Apply” or “application,” as used in reference to a composition, means to apply or spread the composition onto a human keratinous surface such as the skin or hair.

The term “charge density,” as used herein, refers to the ratio of the number of positive charges on a polymer to the molecular weight of said polymer.

“Gel network phase” or “dispersed gel network phase” refers to a lamellar or vesicular solid crystalline phase that includes at least one fatty alcohol, at least one gel network surfactant, and a liquid carrier. The lamellar or vesicular phase can be formed of alternating layers with one layer including the fatty alcohol and the gel network surfactant and the other layer formed of the liquid carrier.

“Solid crystalline” refers to the crystalline structure of the lamellar or vesicular phase at ambient temperatures caused by the phase being below its melt transition temperature. For example, the melt transition temperature of the lamellar or vesicular phase may be 30° C. or more (i.e., slightly above room temperature). The melt transition temperature can be measured through differential scanning calorimetry, which is conventional measurement method known to those skilled in the art.

The term “molecular weight” or “Molecular weight” refers to the weight average molecular weight unless otherwise stated. Molecular weight is measured using industry standard method, gel permeation chromatography (“GPC”).

The term “polymer,” as used herein, includes materials whether made by polymerization of one type of monomer or made by two (i.e., copolymers) or more types of monomers.

“Substantially free of” means a composition or ingredient comprises 3% or less (e.g., 1% or less, 0.5% or less, 0.25% or less) of a subject material, by weight of the composition or ingredient. The term “substantially free” as used herein may also mean that the specific material is not added to the composition but may still be present in small quantities in a raw material that is included in the composition.

The term “suitable for application to human hair,” as used herein, means that the compositions or components thereof so described are acceptable for use in contact with human hair and the scalp and skin without undue toxicity, incompatibility, instability, allergic response, and the like.

“Sulfated surfactants” means surfactants that contain a sulfate moiety. Some non-limiting examples of sulfated surfactants are sodium lauryl sulfate, sodium laureth sulfate, ammonium lauryl sulfate, and ammonium laureth sulfate.

The term “water soluble,” as used herein, means that the material is soluble in water in the present composition. In general, the material should be soluble at 25° C. at a concentration of 0.1% by weight of the water solvent, alternatively at 1%, alternatively at 5%, and alternatively at 15%.

### Conditioning Shampoo Composition

As used herein, the term “dispersed gel network” or “gel network” refers to a lamellar or vesicular solid crystalline phase which comprises at least one fatty alcohol, at least one gel network surfactant, and water and/or other suitable solvents. This dispersed gel network is further combined with a shampoo base comprising a surfactant system, polyvinyl alcohol, one or more cationic polymers, and a liquid carrier to form a conditioning shampoo composition.

### Dispersed Gel Network Phase

The shampoo composition may include a dispersed gel network phase to provide a suitable cleaning benefit to the composition in combination with the detersive surfactant. The shampoo composition can contain 1% to 12% dispersed gel network phase, alternatively 1% to 7% dispersed gel network phase, alternatively 1.25% to 6%, alternatively 1.5% to 5%, and alternatively 2% to 4%.

Suitable dispersed gel networks can be formed by combining a fatty alcohol and a gel network surfactant in a suitable ratio and heating the dispersion to a temperature above the melting point of the fatty alcohol. During the mixing process, the fatty alcohol melts allowing the gel network surfactant to partition and bring water into the fatty alcohol. Mixing of the gel network surfactant and fatty alcohols also changes the isotropic fatty alcohol drops into liquid crystalline phase drops. When the mixture is subsequently cooled below the melt transition temperature of the fatty alcohols, the liquid crystal phase is converted into a solid crystalline gel network. Additional details of suitable gel networks are described in G. M. Eccleston, “Functions of Mixed Emulsifiers and Emulsifying Waxes in Dermatological Lotions and Creams”, Colloids and Surfaces A: Physiochem. and Eng. Aspects 123-124 (1997) 169-182; and by G. M Eccleston, “The Microstructure of Semisolid Creams”, Pharmacy International, Vol. 7, 63-70 (1986), each of which is incorporated by reference herein.

In some aspects, it may be desirable to pre-form the gel network phase, which means that at least fifty percent of the mixture of the fatty alcohol, gel network surfactant, and liquid carrier are in a substantially solid crystalline phase prior to addition to the other components of the shampoo composition. When the dispersed gel network is pre-formed, the gel network component can be prepared as a separate pre-mix, which, after being cooled, can be subsequently incorporated with a detersive surfactant and any other components of a shampoo composition. While not intending to be limited by theory, it is believed that incorporation of a pre-formed gel network component with the detersive surfactant and other components of the shampoo composition allows the formation of a substantially equilibrated lamellar dispersion (“ELD”) in the final composition. The ELD is a dispersed lamellar or vesicular phase resulting from the pre-formed gel network component substantially equilibrating with the detersive surfactants, carrier, and other optional components of a shampoo composition. This equilibration occurs upon incorporation of the pre-formed gel network component with the other components of a shampoo composition and can be effectively complete within 24 hours after incorporation. The ELD does not form if the components which comprise the gel network component (i.e., the fatty alcohol, the gel network surfactant, and the liquid carrier) are added as individual components together with the other components of the shampoo composition in one mixing step, and not as a separate pre-formed gel network component.

The presence of a gel network in the pre-mix and in a shampoo composition can be confirmed by means known to one of skill in the art. For example, X-ray analysis, optical microscopy, electron microscopy, and differential scanning calorimetry can be used to identify a gel network. A suitable x-ray analysis is described in U.S. Patent App. Publication No. 2006/0024256 which is hereby incorporated by reference.

In some aspects, the scale size of the dispersed gel network in a shampoo composition can range 10 nm to 500 nm (e.g., 0.5 μm to 10 μm or 10 μm to 150 μm).

The scale size distribution of the dispersed gel network in a shampoo composition can be measured with a laser light scattering technique using a Horiba model LA 910 Laser Scattering Particle Size Distribution Analyzer (Horiba Instruments, Inc. Irvine California, USA). The scale size distribution in a shampoo composition can be measured by combining 1.75 g of the shampoo composition with 30 mL of 3% NH₄Cl, 20 mL of 2% Na₂HPO₄ 7H₂O, and 10 mL of 1% laureth-7 to form a mixture. This mixture is then stirred for 5 minutes. As appropriate for the individual Horiba instrument being used, samples in the range of 1 to 40 mL are taken and then injected into the Horiba instrument, which contains 75 mL of 3% NH₄Cl, 50 mL of 2% Na₂HPO₄7H₂O, and 25 mL of 1% laureth-7, until the Horiba instrument reading is between 88-92% T, which is needed for the scale size measurement. Once this is achieved, a measurement is taken after 2 minutes of circulation through the Horiba instrument to provide the scale size measurement. A subsequent measurement is taken using a sample of the shampoo composition which has been heated above the melt transition temperature of all fatty materials present in the shampoo composition to ensure the dispersed gel network is melted. This subsequent measurement allows a scale size distribution to be taken of all of the remaining materials in the shampoo composition, which then can be compared to the scale size distribution of the first sample and assist in the analysis.

### Gel Network Fatty Alcohol

The dispersed gel network phase may include a fatty alcohol (e.g., C10-C40 fatty alcohols) at 0.05% or more by weight of the composition (e.g., 0.05% to 25%, 0.5% to 20%, or 1% to 8%). The fatty alcohol may be straight or branched chain and can be saturated or unsaturated. As can be appreciated, suitable fatty alcohols can be of natural, vegetable, or synthetic origin. In some aspects, it may be desirable to mix several fatty alcohols to provide a dispersed gel network phase with a melt transition temperature of 38° C. or greater such as, for example, a mixture of cetyl alcohol and stearyl alcohol at a ratio of between 20:80 and 80:20. Some non-limiting examples of fatty alcohols that may be suitable for use herein include cetyl alcohol, stearyl alcohol, arachidyl alcohol, behenyl alcohol, C21 fatty alcohol (1-heneicosanol), C23 fatty alcohol (1-tricosanol), C24 fatty alcohol (lignoceryl alcohol, 1-tetracosanol), C26 fatty alcohol (1-hexacosanol), C28 fatty alcohol (1-octacosanol), C30 fatty alcohol (1-triacontanol), C20-40 alcohols (e.g., Performacol® 350 and 425 Alcohols, available from New Phase Technologies), C30-50 alcohols (e.g., Performacol® 550 Alcohol), C40-60 alcohols (e.g., Performacol® 700 Alcohol), or mixtures thereof.

The shampoo compositions may include fatty alcohol and at least a portion, if not most of the fatty alcohol is part of the dispersed gel network phase. The composition can include of at least 1%, alternatively 1% to 8%, alternatively 1.25% to 6%, alternatively 1.5% to 5%, alternatively 2% to 5%, and alternatively 2% to 4%, by weight of the shampoo composition.

In one example, the weight ratio of the fatty alcohol to the gel network surfactant in the gel network component may be greater than 1:9, alternatively 1:5 to 100:1, and alternatively 1:1 to 50:1.

### Gel Network Surfactant

The shampoo compositions may include a gel network surfactant and at least a portion, if not most of the gel network surfactant is part of the dispersed gel network phase. The composition may include a gel network surfactant at 0.01% to 15%, alternatively 0.1% to 10%, and alternatively 0.2% to 5%, by weight of the shampoo composition.

The gel network surfactant can be any suitable anionic, zwitterionic, amphoteric, cationic, nonionic surfactants, or mixtures thereof, as described herein. In some examples, the gel network surfactant is free of or substantially free of sulfates. The detersive surfactant and the gel network surfactant can be independently selected and can be the same or different. In some aspects, the gel network surfactant has a hydrophobic tail group with a chain length of 10 to 40 carbon atoms. The hydrophobic tail group may be alkyl, alkenyl (containing up to 3 double bonds), alkyl aromatic, or branched alkyl. Mixtures of more than one gel network surfactant can also be used. Some non-limiting examples of gel network surfactants are disclosed in U.S. Patent App. Pub. No. 2006/0024256.

### Water or Suitable Solvents

The dispersed gel network phase may also include water or suitable solvents. The water or suitable solvent and the gel network surfactant together contribute to the swelling of the fatty alcohol. This, in turn, leads to the formation and the stability of the gel network. As used herein, the term “suitable solvent” refers to any solvent which can be used in the place of or in combination with water in the formation of the gel network.

The shampoo compositions may comprise water or suitable solvents as part of the pre-formed dispersed gel network component in an amount suitable to achieve a gel network when combined with fatty alcohol and gel network surfactant according to the present invention.

The shampoo compositions may comprise as part of the pre-formed dispersed gel network component at least 0.05% of water or a suitable solvent, by weight of the shampoo composition.

The shampoo compositions may comprise water or a suitable solvent as part of the pre-formed dispersed gel network phase is an amount relative to the amount of fatty alcohol at a weight ratio of at least 1:1.

### Shampoo Base

The dispersed gel network phase can be dispersed in a shampoo base. The shampoo base can comprise a cationic deposition polymer, a surfactant, a co-surfactant, an aqueous carrier, and additional components. The resulting shampoo compositions can be substantially free of silicones. As used here in substantially free of silicone means the level of silicone compound is, if included, 0.1% or less, alternatively 0.05% or less, alternatively 0.01% or less, and alternatively 0%.

### Cationic Deposition Polymer

The shampoo compositions may include a cationic deposition polymer. The cationic deposition polymer is included to effectively enhance deposition of the gel network component. The cationic deposition polymer can comprise any cationic polymer that enhances the deposition of the gel network from the shampoo onto the hair and/or scalp.

The concentration of the deposition aid in the shampoo composition may be sufficient to effectively enhance the deposition of the gel network component and ranges 0.05% to 5%, alternatively 0.075% to 2.5%, alternatively 0.1% to 1.0%, alternatively 0.3% to 0.9%, and alternatively 0.4% to 0.8%, by weight of the shampoo composition.

Additionally, the weight ratio of the amount of cationic deposition polymer to PVOH in the shampoo composition can range 0.4 to 10, alternatively 0.5 to 8, alternatively 0.75 to 5, alternatively 1 to 4.

Suitable cationic deposition polymers may have cationic charge densities of at least 0.4 meq/g, alternatively at least 0.7 meq/g, alternatively at least 1.2 meq/g, alternatively at least 1.5 meq/g, alternatively less than 7 meq/g, and alternatively less than 5 meq/g, at the pH of intended use of the composition. The pH will generally range pH 3 to pH 9, alternatively between pH 4 and pH 8. The “cationic” of a polymer, as that term is used herein, refers to the ratio of the number of positive charges on the polymer to the molecular weight of the polymer. The weight average molecular weight of such suitable cationic polymers will generally be between 10,000 and 10 million, alternatively between 50,000 and 5 million, and alternatively between 100,000 and 3 million.

Suitable cationic polymers for use in the composition include polysaccharide polymers, such as cationic cellulose derivatives and cationic starch derivatives, such as salts of hydroxyethyl cellulose reacted with trimethyl ammonium substituted epoxide. Other suitable cationic polymers include cationic guar gum derivatives, such as guar hydroxypropyltrimonium chloride. Further suitable cationic polymers include galactomannan polymer derivatives having a mannose to galactose ratio of greater than 2:1 on a monomer to monomer basis, such as cassia gum hydroxypropyltrimonium chloride. Particularly suitable cationic deposition polymers include guar hydroxypropyltrimonium chloride.

The cationic guar polymer may be formed from quaternary ammonium compounds. In an embodiment, the quaternary ammonium compounds for forming the cationic guar polymer conform to the general formula 1:

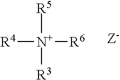

wherein where R3, R4 and R5 are methyl or ethyl groups; R6 is either an epoxyalkyl group of the general formula 2:

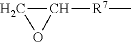

or R6 is a halohydrin group of the general formula 3:

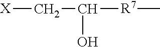

wherein R7 is a C1 to C3 alkylene; X is chlorine or bromine, and Z is an anion such as Cl—, Br—, I— or HSO4—.

In an embodiment, the cationic guar polymer conforms to the general formula 4:

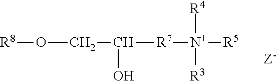

wherein R8 is guar gum; and wherein R4, R5, R6 and R7 are as defined above; and wherein Z is a halogen. In an embodiment, the cationic guar polymer conforms to Formula 5:

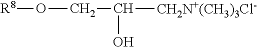

Suitable cationic guar polymers include cationic guar gum derivatives, such as guar hydroxypropyltrimonium chloride. In an embodiment, the cationic guar polymer is a guar hydroxypropyltrimonium chloride. Specific examples of guar hydroxypropyltrimonium chlorides include the Jaguar® series commercially available from Rhone-Poulenc Incorporated, for example Jaguar® C-17, which has a cationic charge density of 0.6 meq/g and a M.Wt. of 2.2 million g/mol and is available from Rhodia Company. Jaguar® C 13S which has a M.Wt. of 2.2 million g/mol and a cationic charge density of 0.8 meq/g (available from Rhodia Company). N-Hance 3196, which has a charge density of 0.7 and a M. Wt. Of 1,100,000 g/mole and is available from ASI. BF-13, which is a borate (boron) free guar of charge density of 1.1 meq/g and M. W.t of 800,000 and BF-17, which is a borate (boron) free guar of charge density of 1.7 meq/g and M. W.t of 800,000 both available from ASI.

A combination of cationic polymers can improve the conditioning and lather of the shampoo composition. Using a cationic polymer with a charge density of 0.4 meq/g to 0.8 meq/g, alternatively 0.7 meq/g in combination with a cationic polymer having a molecular weight greater than 1,000,000 can result in a shampoo composition with both lather stability and creaminess.

In one embodiment, the shampoo composition may comprise a combination of cationic guar and cationic polysaccharide deposition polymers wherein the respective weight ratio of guar to polysaccharide deposition polymers is greater than 2:1, alternatively wherein the weight ratio of guar to polysaccharide deposition polymers is greater than 3:1, and alternatively wherein the weight ratio of guar to polysaccharide deposition polymers is greater than 4:1.

In another embodiment, the shampoo composition may comprise a combination of cationic guar polymers only, wherein one cationic guar has a charge density of 1.7 meq/g and another cationic guar has a molecular weight of 1,100,000 g/molc.

In yet another embodiment, the shampoo composition may comprise a mixture of 3196 guar and BF-17 cationic guar, wherein the weight ratio of these two cationic deposition polymers is 5:1, alternatively 2:1, alternatively 1:1, still alternatively 1:2, and alternatively 2:5 of 3196 to BF-17 respectively.

Additional examples of suitable cationic polymers are described in U.S. application Ser. No. 17/960,862.

### Surfactant System

The shampoo composition can include a surfactant system having one or more anionic detersive surfactants and optionally one or more additional surfactants. Such surfactants can be physically and chemically compatible with the components described herein, or should not otherwise unduly impair product stability, aesthetics, or performance. The shampoo can contain 5% to 50%, alternatively 8% to 30%, alternatively 8% to 20%, and alternatively 10% to 18%, by weight of the composition, surfactant system.

### Anionic Detersive Surfactant

The shampoo composition may comprise one or more anionic detersive surfactants in the shampoo base to provide cleansing performance. The concentration of the anionic surfactants in the shampoo composition can be sufficient to provide the desired cleaning and lather performance, and generally range from 4% to 25%, alternatively 8% to 20%, alternatively 8% to 15%, and alternatively 10% to 14%, by weight of the composition.

Additional anionic surfactants suitable for use herein include alkyl and alkyl ether sulfates of the formula ROSO₃M and RO(C₂H₄O)ₓSO₃M, wherein R is alkyl or alkenyl of 8 to 18 carbon atoms, x is 1 to 10, and M is a water-soluble cation such as ammonium, sodium, potassium, and triethanolamine cation or salts of the divalent magnesium ion with two anionic surfactant anions. The alkyl ether sulfates may be made as condensation products of ethylene oxide and monohydric alcohols having 8 to 24 carbon atoms. The alcohols can be derived from fats such as coconut oil, palm oil, palm kernel oil, or tallow, or can be synthetic.

Other suitable anionic surfactants include water-soluble salts of the organic, sulfonic acids of the general formula [R¹—SO₃M]. R¹ being a straight chain aliphatic hydrocarbon radical having from 13 to 17 carbon atoms, alternatively from 13 to 15 carbon atoms. M is a water soluble cation such as ammonium, sodium, potassium, and triethanolamine cation or salts of the divalent magnesium ion with two anionic surfactant anions. These materials are produced by the reaction of SO₂ and O₂ with suitable chain length normal paraffins (C₁₄-C₁₇) and are sold commercially as sodium paraffin sulfonates.

In one example, the anionic detersive surfactant may be a combination of sodium lauryl sulfate and sodium laureth-n sulfate.

Examples of detersive anionic surfactants suitable can include, but are not limited to, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium trideceth sulfate, sodium tridecyl sulfate, sodium methyl lauroyl taurate, sodium methyl cocoyl taurate, sodium lauroyl isethionate, sodium cocoyl isethionate, sodium laurethsulfosuccinate, sodium laurylsulfosuccinate, sodium tridecyl benzene sulfonate, sodium dodecyl benzene sulfonate, or mixtures thereof.

In some examples, the composition can include one or more sulfate-free anionic surfactants such as sodium, ammonium or potassium salts of isethionates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof.

Some particularly suitable examples of sulfate-free anionic surfactants can include isethionates, sarcosinates, sulfonates or mixtures thereof.

Suitable isethionate surfactants can include the reaction product of fatty acids esterified with iscthionic acid and neutralized with sodium hydroxide. Suitable fatty acids for isethionate surfactants can be derived from coconut oil or palm kernel oil including amides of methyl tauride. Non-limiting examples of isethionates can be selected from the group consisting of sodium lauroyl methyl isethionate, sodium cocoyl isethionate, ammonium cocoyl isethionate, sodium hydrogenated cocoyl methyl isethionate, sodium lauroyl isethionate, sodium cocoyl methyl isethionate, sodium myristoyl isethionate, sodium oleoyl isethionate, sodium oleyl methyl isethionate, sodium palm kerneloyl isethionate, sodium stearoyl methyl isethionate, or mixtures thereof.

The amino acid based anionic surfactant can be a sarcosinate, for instance an acyl sarcosinate. Non-limiting examples of sarcosinates can be selected from the group consisting of sodium lauroyl sarcosinate, sodium cocoyl sarcosinate, sodium myristoyl sarcosinate, TEA-cocoyl sarcosinate, ammonium cocoyl sarcosinate, ammonium lauroyl sarcosinate, dimer dilinoleyl bis-lauroylglutamate/lauroylsarcosinate, disodium lauroamphodiacetate lauroyl sarcosinate, isopropyl lauroyl sarcosinate, potassium cocoyl sarcosinate, potassium lauroyl sarcosinate, sodium cocoyl sarcosinate, sodium lauroyl sarcosinate, sodium myristoyl sarcosinate, sodium oleoyl sarcosinate, sodium palmitoyl sarcosinate, TEA-cocoyl sarcosinate, TEA-lauroyl sarcosinate, TEA-oleoyl sarcosinate, TEA-palm kernel sarcosinate, and combinations thereof.

Non-limiting examples of sulfonates can include alpha olefin sulfonates, linear alkylbenzene sulfonates, sodium laurylglucosides hydroxypropylsulfonate and combinations thereof.

### Additional Surfactants

The shampoo compositions may, optionally, include one or more additional surfactants. The additional surfactants may be selected from cationic surfactants such as polyquaternium surfactants; amphoteric/zwitterionic surfactants such as those surfactants broadly described as derivatives of aliphatic secondary and tertiary amines in which one of the aliphatic substituents contains from 8 to 18 carbon atoms and one aliphatic substituent contains an anionic group such as a carboxy, sulfonate, phosphate, or phosphonate group; non-ionic surfactants such as the polyethylene oxide condensates of alkyl phenols; and combinations of these. Some non-limiting examples of the optional surfactants described above are disclosed in US 20190105246, US 20180098923, U.S. Pat. No. 9,271,908, WO 2020/016097, and Mccutcheon's Emulsifiers and Detergents, 2019, MC Publishing Co.

If present, the composition can contain 1% to 10%, alternatively 1.5% to 5%, and alternatively 2% to 4%, by weight of the composition, additional surfactant.

Some suitable examples of additional surfactants include amphoteric surfactants selected from cocoamphoacetates, cocoamphodiacetates, lauroamphoacetates, lauroamphodiacetates, amidobetaines, amidosulfobetaines or mixtures thereof.

Additional examples of amphoteric surfactants can be selected from the group consisting of betaines, sultaines, hydroxysultanes, amphohydroxypropyl sulfonates, alkyl amphoactates, alkyl amphodiacetates, alkyl amphopropionates and combination thereof.

Examples of betaine amphoteric surfactants can include coco dimethyl carboxymethyl betaine, cocoamidopropyl betaine (CAPB), cocobetaine, lauryl amidopropyl betaine (LAPB), oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, cetyl betaine, or mixtures thereof. Examples of sulfobetaines can include coco dimethyl sulfopropyl betaine, stearyl dimethyl sulfopropyl betaine, lauryl dimethyl sulfoethyl betaine, lauryl bis-(2-hydroxyethyl) sulfopropyl betaine or mixtures thereof.

The shampoo composition may further comprise additional surfactants for use in combination with the anionic detersive surfactant component described herein. Suitable additional surfactants include cationic and nonionic surfactants.

Non-limiting examples of other anionic, zwitterionic, amphoteric, cationic, nonionic, or optional additional surfactants suitable for use in the compositions are described in Mccutcheon's, Emulsifiers and Detergents, 1989 Annual, published by M. C. Publishing Co., and U.S. Pat. Nos. 3,929,678; 2,658,072; 2,438,091; 2,528,378; 5,104,646 and 5,106,609.

### Liquid Carrier

The shampoo composition can desirably be in the form of pourable liquid under ambient conditions. Inclusion of an appropriate quantity of a liquid carrier can facilitate the formation of a shampoo composition having an appropriate viscosity and rheology. A shampoo composition can include, by weight of the composition, 20% to 95%, by weight, of a liquid carrier, and alternatively 60% to 85%, by weight, of a liquid carrier. A shampoo composition can include ≥50%, alternatively ≥60%, alternatively ≥70%, alternatively ≥75%, by weight, of a liquid carrier. The liquid carrier can be an aqueous carrier such as water.

### Other Optional Ingredients

The shampoo compositions described herein may include a variety of optional ingredients to tailor the properties and characteristics of the composition, as desired. The optional ingredients may be well known materials that are commonly included in compositions of the type. The options ingredients can be physically and chemically compatible with the essential components of the shampoo compositions and should not otherwise unduly impair the stability, aesthetics, or performance of the composition. Individual concentrations of optional components can generally range from 0.001% to 10%, by weight of a shampoo composition.

Some non-limiting examples of other optional ingredients that can be included in the shampoo compositions herein include: co-surfactants, deposition aids, cationic polymers, conditioning agents (including hydrocarbon oils, fatty esters, silicones), anti-dandruff agents, anti-microbial agents, suspending agents, viscosity modifiers, dyes, pigments, nonvolatile solvents or diluents (water soluble and insoluble), pearlescent aids, foam boosters, pediculocides, pH adjusting agents, perfumes, preservatives, chelants, proteins, vitamins, amino acids, skin active agents, sunscreens, UV absorbers, stabilizers, and combinations of these.

### Method of Making a Personal Care Composition

The compositions described herein can be made using conventional methods. In some aspects, the composition may be made by: (a) combining a gel network fatty alcohol, a gel network surfactant, and water at a temperature sufficient to allow partitioning of the gel network surfactant and the water into the fatty alcohol to form a pre-mix; (b) cooling the pre-mix below the chain melt temperature of the fatty alcohol to form a gel network; (c) adding the gel network to a shampoo base that includes one or more detersive surfactants, a cationic polymer, polyvinyl alcohol, and a liquid carrier to form a conditioning shampoo composition which includes a dispersed gel network phase having a melt transition temperature of at least 38° C.

In some aspects, the gel network phase can be prepared by heating the fatty alcohol, the gel network surfactant, and water to a level in the range of 75° C. to 90° C. and mixing. This mixture can be cooled to 27-35° C. (e.g., by passing the mixture through a heat exchanger). As a result of this cooling step, at least fifty percent of the mixture of the fatty alcohol and the gel network surfactant crystallize to form a crystalline gel network.

Other methods of preparing the gel network phase include sonication and/or milling of the fatty alcohol, the gel network surfactant, and water, while these components are heated, to reduce the particle size of the dispersed gel network phase. This results in an increase in surface area of the gel network phase, which allows the gel network surfactant and the water to swell the gel network phase. Another variation in preparing the gel network includes heating and mixing the fatty alcohol and the gel network surfactant first, and then adding that mixture to the water.

### Method of Use

The shampoo compositions can be used in a conventional manner for cleansing and conditioning hair. Effective amounts of the composition for use generally range from 1 g to 50 g (e.g., 1 g to 20 g). Generally, a method of treating hair can include wetting the hair, squirting an effective amount of a liquid conditioning shampoo composition into a user's palm, applying the composition to the hair by massaging the composition into the hair and scalp until it lathers, working the composition through the hair, and then the composition can be rinsed off.

In some aspects, the method for treating the hair or skin can include the steps of: (a) wetting the hair or skin with water; (b) applying an effective amount of the personal care composition to the hair or skin, and (c) rinsing the applied areas of skin or hair with water. These steps can be repeated as many times as desired to achieve the desired cleansing and conditioning benefit.

### Test Methods

### Cone/Plate Viscosity Measurement

The viscosities of the examples are measured by a Cone/Plate Controlled Stress Brookfield Rheometer R/S Plus, by Brookfield Engineering Laboratories, Stoughton, MA. The cone used (Spindle C-75-1) has a diameter of 75 mm and 1° angle. The liquid viscosity is determined using a steady state flow experiment at constant shear rate of 2 s-1 and at temperature of 26.7° C. The sample size is 2.5 ml to 3 ml and the total measurement reading time is 3 minutes.

### PVOH Viscosity Method

The viscosity of the PVOH is measured as a 4% aqueous solution by a Brookfield DV-II+pro cup and bob viscometer with ultra-low viscosity adaptor. The measurement geometry is cylindrical type 0 rotor. The sample temperature is 20° C. The shear rate is adjusted to accommodate PVOH variants with different viscosities while maintaining a torque within the range of 60-80%.

### Hair Texture Analysis Method

### Hair Substrate

Round ponytail hair tresses of general population, Chinese hair measuring 4 grams in weight and 8 inches in length were purchased from International Hair Importers & Products Inc., 87-29 Myrtle Ave., Glendale, NY 11385 and used in the hair treatment procedure that follows. Each test product was applied to 3 separate hair tresses (n=3).

### Hair Treatment

0.4 grams (0.1 g/gram hair) of a conditioning shampoo (e.g., Composition A) was applied to a hair tress that was first prewetted with 100-105 OF water and then squeegeed to remove any excess water. The product was applied down the hair tress in a zig-zag pattern, equally to the front and back of the hair tress (0.2 grams per side). The hair tress was then brushed, using a Goody Brush with large, stiff, plastic bristles, alternating down the front and back for 30 strokes at approximately 1 stroke per second for a total of 30 seconds. The tress was then rinsed using 100-105° F. water for 30 seconds while milking the switch at approximately 1 stroke per second. The tress was squeezed to remove any excess water. The test product was then reapplied to the hair tress a second time following the same procedure described above and then rinsed from the hair tress.

### Measurement of the Combing Force

The force required to comb a hair tress after treatment (wet and dry) was measured using the Texture Analyzer TA-XT Plus (manufactured by Stable Micro Systems), Instron 5542 (manufactured by Instron) or equivalent force measurement device. This method is an industry standard method for measuring wet/dry hair combing forces, disclosed by TRI Princeton.

The hair tress is placed within the holder of the Texture Analyzer, fixed at the root end of the hair tress. The hair tress is positioned within the combs in series and then pulled through the combs by the Texture Analyzer, while the average force to pull the tress through each comb is recorded (=1 combing stroke). The hair tress is disengaged from the combs and returned to its pre-combing position. The hair tress is then combed 9 more times using the same combing procedure. The outputs include work (gram force, gf) to detangle and combing at tips (resistance at tips).

### EXAMPLES

The following data and examples are provided to help illustrate the haircare compositions described herein. The exemplified compositions are given solely for the purpose of illustration and are not to be construed as limitations of the present disclosure, as many variations thereof are possible without departing from the spirit and scope of the disclosure. All parts, percentages, and ratios herein are by weight unless otherwise specified. The levels given reflect the weight percent of the active material, unless otherwise specified. A level of perfume and/or preservatives may also be included in the following examples.

Table 1 shows comparative shampoo compositions and Table 3 shows inventive shampoo compositions. Examples C1-C5 and Ex. 1-3 and 7 were made using conventional methods and Ex.4-6 and 8 could be made using conventional methods. The detangling and resistance at tips were determined according to the methods described herein.

TABLE 1
Gel Network Premix

| | (wt. %) |
|---|---|
| Sodium Laureth-1 Sulfate | 11.00 |
| Stearyl Alcohol | 7.71 |
| Cetyl Alcohol | 4.29 |
| Water | QS |

TABLE 2
Comparative Examples

| | C1 | C2 | C3 | C4 | C5 |
|---|---|---|---|---|---|
| Sodium Laureth | 11.60 | 11.60 | 11.60 | 11.60 | 11.60 |
| Sulfate ⁽¹⁾ | | | | | |
| Sodium Lauryl | 1.40 | 1.40 | 1.40 | 1.40 | 1.40 |
| Sulfate | | | | | |
| Cocamidopropyl | 2.00 | 2.00 | 2.00 | 2.00 | 2.00 |
| Betaine | | | | | |
| Polyvinyl | 0.28 | | 0.56 | 0.42 | |
| Alcohol 3-80 ⁽²⁾ | | | | | |
| Polyvinyl | 0.12 | | 0.24 | 0.18 | |
| Alcohol 32-80 ⁽³⁾ | | | | | |
| Guar Hydroxypropyl | 0.40 | 0.80 | | 0.20 | |
| Trimonium Chloride ⁽⁵⁾ | | | | | |
| Guar Hydroxypropyl | | | | | 0.40 |
| Trimonium Chloride ⁽⁶⁾ | | | | | |
| Gel Network Premix | | 2.30 | 2.30 | 2.30 | 1.00 |
| from Table 1 | | | | | |
| Hydrogenated | 0.06 | 0.06 | 0.06 | 0.06 | |
| Castor Oil ⁽⁹⁾ | | | | | |
| Ethylene Glycol | | | | | 1.50 |
| Distearate ⁽¹⁰⁾ | | | | | |
| Dimethicone ⁽¹²⁾ | | | | | 3.00 |
| Citric Acid/Sodium | Adjust to PH ~5-7 | | | | |
| Citrate Dihydrate/ | | | | | |
| HCl/NaOH | | | | | |
| Sodium Chloride/ | Adjust to viscosity of ~4-14 Pa · s | | | | |
| Ammonium | | | | | |
| Xylene Sulfonate | | | | | |
| Water, Fragrance, | Q.S. to 100 | | | | |
| Preservatives | | | | | |
| Comparative Test Group | Group 1 | | | | Group 2 |
| Detangling | 176 | 154 | 210 | 200 | 184 |
| (avg) | | | | | |
| Detangling | 24 | 17 | 22 | 30 | 26 |
| (stdev) | | | | | |
| Resistance at Tips | 130 | 100 | 141 | 134 | 171 |
| (avg) | | | | | |
| Resistance at Tips | 29 | 13 | 34 | 27 | 34 |
| (stdev) | | | | | |

TABLE 3
Examples

| | Ex. 1 | Ex. 2 | Ex. 3 | Ex. 4 | Ex. 5 | Ex. 6 | Ex. 7 | Ex. 8 |
|---|---|---|---|---|---|---|---|---|
| Sodium Laureth Sulfate ⁽¹⁾ | 11.60 | 11.60 | 11.60 | | 12.00 | | 11.60 | 11.60 |
| Sodium Lauryl Sulfate | 1.40 | 1.40 | 1.40 | 14.00 | | | 1.40 | 1.40 |
| Sodium Cocoyl Isethionate | | | | | | 6.00 | | |
| Sodium Lauryl Sarcosinate | | | | | | 2.50 | | |
| Cocamidopropyl Betaine | 2.00 | 2.00 | 2.00 | | 4.00 | 4.40 | 2.00 | 2.00 |
| Lauramidopropyl Betaine | | | | 2.00 | | 4.60 | | |
| Cocamide MEA | | | | 1.00 | | | | |
| Polyvinyl Alcohol 3-80 ⁽²⁾ | 0.35 | 0.14 | | | | | 0.28 | |
| Polyvinyl Alcohol 32-80 ⁽³⁾ | 0.15 | 0.06 | | 0.60 | | | 0.12 | 0.20 |
| Polyvinyl Alcohol 3-98 ⁽⁴⁾ | | | 0.20 | | 0.40 | 0.50 | | |
| Guar Hydroxypropyl | 0.40 | 0.40 | 0.40 | 0.40 | 0.10 | | 0.40 | 0.80 |
| Trimonium Chloride ⁽⁵⁾ | | | | | | | | |
| Polyquaternium-10 ⁽⁷⁾ | | | | | 0.10 | 0.25 | | |
| Polyquaternium-6 ⁽⁸⁾ | | | | 0.10 | | | | |
| Gel Network Premix from | 2.30 | 2.30 | 2.30 | 2.00 | 4.80 | 2.30 | 2.30 | 2.30 |
| Table 1 | | | | | | | | |
| Hydrogenated Castor Oil ⁽⁹⁾ | 0.06 | 0.06 | 0.06 | 0.06 | 0.06 | | 0.06 | 0.06 |
| Acrylates Copolymer ⁽¹¹⁾ | | | | | | 1.00 | | |
| Citric Acid/Sodium | Adjust to pH ~5-7 | | | | | | | |
| Citrate Dihydrate/ | | | | | | | | |
| HCl/NaOH | | | | | | | | |
| Sodium Chloride/ | Adjust to viscosity of ~4-14 Pa · s | | | | | | | |
| Ammonium Xylene | | | | | | | | |
| Sulfonate | | | | | | | | |
| Water, Fragrance, | Q.S. to 100 | | | | | | | |
| Preservatives | | | | | | | | |
| | Dry combing measures (gram force, gf)* | | | | | | | |
| Comparative Test Group | Group 2 | Group 1 | | | | | | |
| Detangling (avg) | 131 | 117 | | | | | 79 | |
| Detangling (stdev) | 9 | 12 | | | | | 6 | |
| Resistance at Tips (avg) | 91 | 78 | | | | | 53 | |
| Resistance at Tips (stdev) | 14 | 10 | | | | | 6 | |

*Values represent average (mean) gram force (gf) values for combing strokes 2 thru 6 (of a 10-stroke test), using a sample size of n = 3 hair tresses per product. Lower values represent less combing force and higher conditioning, which is preferred for the invention herein.

### Tradenames and Suppliers for Table 2 to Table 3:

(1) Sodium Laureth-n Sulfate, where n ≥1 and ≤3
(2) PVOH 3-80 (Kuraray Poval®)
(3) PVOH 32-80 (Kuraray Poval®)
(4) PVOH 3-98 (Kuraray Poval®)
(5)N-Hance® 3196 (Ashland™)
(6) Jaguar® C500 (Solvay®)
(7) Polymer JR30M (Dow®)
(8) Mirapol® 100S (SNF)
(9) Thixcin® R (Elementis®)
(10) EGDS Purified (Evonik®)
(11) Rheocare®TTA (BASF®)
(12) 330M Silicone (Momentive®)

Examples 1, 2, and 7, on average, had improved detangling and wet combing, as compared to Comparative Examples C1-5. It is interesting to note that C5 contains cationic polymer, gel network, and 3% silicone and Examples 1, 2, and 7 are silicone-free with 0.2-0.5% PVOH and out-performed C5. It is believed that the examples in Table 3 would have consumer acceptable detangling and wet combing.

The data in Table 4, below, shows the results from consumer tests with Chinese female consumers ages 18-45. Three separate test groups with eight consumers in each group used the test product in place of their normal shampoo product for one week. Consumers then gave each product an overall rating using a sliding rating scale from 1 (did not like this shampoo) to 100 (love this shampoo). The scores for each test group were averaged (mean) and then the scores were normalized to enable comparison between the test groups. Individual product test scores were normalized with the highest test score (94— Test Group 2) obtained for the silicone control shampoo (C3). It was found that the non-silicone shampoo (Example 1) delighted consumers about the same as the silicone containing control C5.

TABLE 1

| | Test Group 1 | Test Group 2 | Test Group 3 |
|---|---|---|---|
| Silicone Control | 79/94 | 94/94 | 86/94 |
| Shampoo | | | |
| (C5, Table 2) | | | |
| Non-Silicone | | | 80/87 |
| Shampoo | | | |
| (Ex.1, Table 3) | | | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A conditioning shampoo, comprising:
a) about 4% to about 25% of a detersive surfactant;
b) about 0.05% to about 5% of a polyvinyl alcohol;
c) about 1% to about 8% of a fatty alcohol;
d) about 0.01% to about 15% of a gel network surfactant chosen from anionic surfactants, cationic surfactants, zwitterionic surfactants, non-ionic surfactants, and mixtures thereof;
e) about 60% to about 85% of a liquid carrier; and
f) a dispersed gel network phase, comprising:
i) at least a portion of the fatty alcohol;
ii) at least a portion of the gel network surfactant; and
iii) at least a portion of the liquid carrier, wherein the detersive surfactant and the gel network surfactant are the same or different, the composition is substantially free of silicone, and the composition is a liquid.

2. The conditioning shampoo of claim 1, wherein the polyvinyl alcohol has a degree of hydrolysis ≥80%.

3. The conditioning shampoo of claim 2, wherein the polyvinyl alcohol has a degree of hydrolysis ≥90%.

4. The conditioning shampoo of claim 1, wherein the polyvinyl alcohol has a viscosity about 4 cP to about 32 cP, as measured as a 4% aqueous solution at 20° C.

5. The conditioning shampoo of claim 1, wherein the fatty alcohol is chosen from cetyl alcohol, stearyl alcohol, or mixtures thereof.

6. The conditioning shampoo of claim 1, wherein the composition is free of silicone.

7. The conditioning shampoo of claim 1, wherein the conditioning shampoo provides an average detangling of less than 210 gf according to the Hair Texture Analysis Method.

8. The conditioning shampoo of claim 7, wherein the conditioning shampoo provides an average detangling of about 45 gf to about 180 gf.

9. The conditioning shampoo of claim 1, wherein the conditioning shampoo provides a resistance at tips of less than 200 gf according to the Hair Texture Analysis Method.

10. The conditioning shampoo of claim 9, wherein the conditioning shampoo provides a resistance at tips of less than 150 gf.

11. The conditioning shampoo of claim 1, wherein the conditioning shampoo is free of sulfated surfactants.

12. The conditioning shampoo of claim 1, further comprising a cationic deposition polymer.

13. The conditioning shampoo of claim 12, wherein a weight ratio of cationic deposition polymer to polyvinyl alcohol is about 0.4:1 to about 10:1.

14. The conditioning shampoo of claim 13, wherein the weight ratio of cationic deposition polymer to polyvinyl alcohol is about 0.5:1 to about 8:1.

15. The conditioning shampoo of claim 14, wherein the weight ratio of cationic deposition polymer to polyvinyl alcohol is about 1:1 to about 4:1.

16. The conditioning shampoo of claim 1, further comprising a co-surfactant chosen from amphoteric surfactants, zwitterionic surfactants, nonionic surfactants, and mixtures thereof.

17. The conditioning shampoo of claim 16, wherein the composition comprises a zwitterionic surfactant chosen from a betaine, a sultaine, and mixtures thereof.

18. The conditioning shampoo of claim 1, wherein the composition has a viscosity of about 2000 cP to about 23,000 cP according to the Cone/Plate Viscosity Measurement.
